(19) United States
(12) Patent Application Publication
(10) Pub. No.: US 2026/0273220 A1
(43) Pub. Date: Sep. 17, 2026

(54) Intermittent-Catheter Assembly and Methods Thereof

(71) Applicant: C. R. Bard, Inc., Franklin Lakes, NJ (US)

(72) Inventors: James David Hughett, SR., Monroe, GA (US); Pooja Bhuvanesh Kulkarni, Pune (IN)

(21) Appl. No.: 19/659,980

(22) Filed: Apr. 27, 2026

Related U.S. Application Data
(63) Continuation of application No. 18/025,875, filed on Sep. 10, 2021, now Pat. No. 12,611,519.
(60) Provisional application No. 63/077,469, filed on Sep. 11, 2020.

(51) Int. Cl. A61M 25/00

(52) U.S. Cl. CPC A61M 25/0017

(57) ABSTRACT

An intermittent-catheter assembly can include an intermittent catheter and a catheter housing. The intermittent catheter includes a funnel and a catheter tube fluidly coupled to the funnel. The catheter housing can include the intermittent catheter disposed in the catheter housing while in a packaged state of the intermittent-catheter assembly. The catheter housing can include an inner sleeve and an outer sleeve. The inner sleeve can include a longitudinal cavity containing a majority of the intermittent catheter in the packaged state of the intermittent-catheter assembly. The outer sleeve can be slidably disposed over the inner sleeve. The catheter housing can be configured to expose the intermittent catheter for removal from the catheter housing when the outer sleeve is grasped and slid toward an exposed end of the inner sleeve in opposition to a force applied to the exposed end of the inner sleeve.

### PRIORITY

This application is a continuation of U.S. patent application Ser. No. 18/025,875, filed Mar. 10, 2023, now U.S. Pat. No. 12,611,519, which is a U.S. national stage of International Patent Application No. PCT/US2021/049867, filed Sep. 10, 2021, which claims the benefit of priority to U.S. Provisional Application No. 63/077,469, filed Sep. 11, 2020, each of which is incorporated by reference in its entirety into this application.

### BACKGROUND

Users of urinary catheters such as intermittent catheters self-catheterize four to six times a day. As such, a simple-to-use intermittent catheter that ensures sterility before use and facilitates cleanliness after use is needed.

Disclosed herein are intermittent-catheter assemblies and methods thereof that address the foregoing.

### SUMMARY

Disclosed herein is an intermittent-catheter assembly including, in some embodiments, an intermittent catheter and a catheter housing. The intermittent catheter includes a funnel and a catheter tube fluidly coupled to the funnel. The catheter housing includes the intermittent catheter disposed in the catheter housing while in a packaged state of the intermittent-catheter assembly. The catheter housing includes an inner sleeve and an outer sleeve. The inner sleeve includes a longitudinal cavity containing a majority of the intermittent catheter in the packaged state of the intermittent-catheter assembly. The outer sleeve is slidably disposed over the inner sleeve. The catheter housing is configured to expose the intermittent catheter for removal from the catheter housing when the outer sleeve is grasped and slid toward an exposed end of the inner sleeve in opposition to a force applied to the exposed end of the inner sleeve.

In some embodiments, the inner sleeve includes longitudinal ribs extending into the longitudinal cavity. The ribs stabilize the intermittent catheter in the inner sleeve in the packaged state of the intermittent-catheter assembly.

In some embodiments, the catheter housing further includes a displaceable cap sealing an opening of the outer sleeve opposite the exposed end of the inner sleeve. The cap seals the opening of the outer sleeve and maintains sterility of the intermittent catheter in the packaged state of the intermittent-catheter assembly.

In some embodiments, the cap sits in a seat formed within the opening of the outer sleeve, the cap tethered to the inner sleeve by a tether.

In some embodiments, the tether is a polymeric ribbon. The ribbon includes an opening configured to weaken a structural integrity of the ribbon around the opening such that the ribbon and the cap tethered thereto bend away from a centerline of the catheter housing as the outer sleeve is slid toward the exposed end of the inner sleeve. So configured, the ribbon facilitates access to the intermittent catheter.

In some embodiments, the cap includes an annular gasket disposed in a recess around a rim of the cap. The gasket sits between the cap and the outer sleeve securing the cap in the outer sleeve in the packaged state of the intermittent-catheter assembly.

In some embodiments, the cap includes a funnel insert along a centerline of the cap. The funnel insert is inserted into the funnel of the intermittent catheter in the packaged state of the intermittent-catheter assembly, which stabilizes the intermittent catheter in the inner sleeve.

In some embodiments, the cap sits on a seat formed around the opening of the outer sleeve in the packaged state of the intermittent-catheter assembly. The cap is coupled to the outer sleeve by a living hinge.

In some embodiments, the cap and the outer sleeve include complementary snap-fit features securing the cap on the outer sleeve in the packaged state of the intermittent-catheter assembly.

In some embodiments, the inner sleeve includes a longitudinal side opening to the longitudinal cavity. The inner sleeve includes an end cap integrated into the inner sleeve opposite the exposed end of the inner sleeve.

In some embodiments, a connecting portion of the inner sleeve is coterminous with ends of the side opening. The connecting portion is molded with a bias such that the connecting portion and the end cap coupled thereto bend away from a centerline of the catheter housing as the outer sleeve is slid toward the exposed end of the inner sleeve. So configured, the connecting portion facilitates access to the intermittent catheter.

In some embodiments, the intermittent-catheter assembly further includes a drainage bag. The drainage bag is fluidly coupled to the funnel for voiding urine into the drainage bag upon catheterization with the intermittent catheter.

Also disclosed herein is an intermittent-catheter assembly including, in some embodiments, an intermittent catheter and a catheter housing. The intermittent catheter includes a funnel and a catheter tube fluidly coupled to the funnel. The catheter housing includes the intermittent catheter disposed in the catheter housing while in a packaged state of the intermittent-catheter assembly. The catheter housing includes a sleeve and a cap. The sleeve includes a longitudinal cavity containing a majority of the intermittent catheter in the packaged state of the intermittent-catheter assembly. The cap is coupled to the sleeve by a living hinge. The cap seals an opening of the sleeve opposite a closed end of the sleeve. The cap seals the opening of the sleeve and maintains sterility of the intermittent catheter in the packaged state of the intermittent-catheter assembly.

In some embodiments, the opening includes a longitudinal extension into the sleeve. The longitudinal extension into the sleeve is configured to expose a longitudinal portion of the funnel when the cap is moved from a closed position in the packaged state of the intermittent-catheter assembly to an open position about the living hinge.

In some embodiments, the cap includes a pull tab coupled to the cap on a same side of the catheter housing as the living hinge. A portion of the pull tab is configured to peel away from the cap toward an opposite side of the catheter housing from the living hinge when the pull tab is initially pulled to extend a lever arm with respect to the living hinge sufficient for subsequently pulling the cap away from the opening of the sleeve by the pull tab.

In some embodiments, the cap includes a push tab extending from an opposite side of the catheter housing from the living hinge. The push tab is configured to extend a lever arm with respect to the living hinge sufficient for pushing the cap away from the opening of the sleeve by the push tab.

In some embodiments, the cap includes a push button. The push button is configured to deform the cap and disengage complementary snap-fit features between the cap and the sleeve for subsequently pushing the cap away from the opening of the sleeve by the push button.

In some embodiments, the intermittent-catheter assembly further includes a drainage bag. The drainage bag is fluidly coupled to the funnel for voiding urine into the drainage bag upon catheterization with the intermittent catheter.

Also disclosed herein is an intermittent-catheter assembly including, in some embodiments, an intermittent catheter and a catheter housing. The intermittent catheter includes a funnel and a catheter tube fluidly coupled to the funnel. The catheter housing includes the intermittent catheter disposed in the catheter housing while in a packaged state of the intermittent-catheter assembly. The catheter housing includes a sleeve and a removable cap. The sleeve includes a longitudinal cavity containing a majority of the intermittent catheter in the packaged state of the intermittent-catheter assembly. The cap includes another longitudinal cavity configured to contain a remainder of the intermittent catheter. The cap seals an opening of the sleeve opposite a closed end of the sleeve. The cap seals the opening of the sleeve and maintains sterility of the intermittent catheter in the packaged state of the intermittent-catheter assembly.

In some embodiments, the sleeve includes longitudinal ribs extending into the longitudinal cavity. The ribs stabilize the intermittent catheter in the sleeve in the packaged state of the intermittent-catheter assembly.

In some embodiments, the sleeve is approximately coextensive with the catheter tube and the cap is approximately coextensive with the funnel.

In some embodiments, the cap and the sleeve include complementary snap-fit features. The snap-fit features secure the cap on the sleeve in the packaged state of the intermittent-catheter assembly.

In some embodiments, the intermittent-catheter assembly further includes shrink-wrap packaging over an entirety of the cap and at least a portion of the sleeve. The packaging includes a pull tab extending from the packaging configured to break open the packaging when the pull tab is pulled.

In some embodiments, the cap includes internal threads about an open-ended portion of the cap and the sleeve includes complementary external threads about an open-ended portion of the sleeve including the opening. The cap includes recesses or ribs around the cap configured to facilitate gripping and screwing the cap off the sleeve.

In some embodiments, the intermittent-catheter assembly further includes a drainage bag. The drainage bag is fluidly coupled to the funnel for voiding urine into the drainage bag upon catheterization with the intermittent catheter.

Also disclosed herein is an intermittent-catheter assembly including, in some embodiments, an intermittent catheter and a catheter housing. The intermittent catheter includes a funnel and a catheter tube fluidly coupled to the funnel. The catheter housing includes the intermittent catheter disposed in the catheter housing while in a packaged state of the intermittent-catheter assembly. The catheter housing includes a sleeve, a reinforcing insert, and an adhesive tab. The sleeve includes a longitudinal cavity containing a majority of the intermittent catheter in the packaged state of the intermittent-catheter assembly. The reinforcing insert is suspended in a longitudinal sleeve gap. The reinforcing insert includes a longitudinal insert gap such that the sleeve gap and the insert gap combine to provide major-side openings for grasping the funnel and removing the intermittent catheter from the catheter housing. The adhesive tab covers the major-side openings of the catheter housing.

In some embodiments, the reinforcing insert includes major-side protrusions from which the reinforcing insert is suspended in the sleeve gap.

In some embodiments, the reinforcing insert includes minor-side protrusions from which the reinforcing insert is suspended in the sleeve gap.

In some embodiments, the intermittent-catheter assembly further includes a drainage bag. The drainage bag is fluidly coupled to the funnel for voiding urine into the drainage bag upon catheterization with the intermittent catheter.

Also disclosed herein is an intermittent-catheter assembly including, in some embodiments, an intermittent catheter and a catheter housing. The intermittent catheter includes a funnel and a catheter tube fluidly coupled to the funnel. The catheter housing includes the intermittent catheter disposed in the catheter housing while in a packaged state of the intermittent-catheter assembly for maintaining sterility of the intermittent catheter. The catheter housing includes an end piece, a collapsible sheath, a pull tab, and a removable cap. The collapsible sheath includes a distal portion coupled to the end piece and a proximal portion coupled to the funnel. An entirety of the catheter tube is disposed in the collapsible sheath in the packaged state of the intermittent-catheter assembly. The pull tab seals a distal opening of the end piece in the packaged state of the intermittent-catheter assembly. The removable cap seals a proximal opening of the funnel in the packaged state of the intermittent-catheter assembly.

In some embodiments, the intermittent-catheter assembly further includes a drainage bag. The drainage bag is fluidly coupled to the funnel for voiding urine into the drainage bag upon catheterization with the intermittent catheter.

Also disclosed herein is an intermittent-catheter assembly including, in some embodiments, an intermittent catheter and a catheter housing. The intermittent catheter includes a funnel and a catheter tube fluidly coupled to the funnel. The catheter housing includes the intermittent catheter disposed in the catheter housing while in a packaged state of the intermittent-catheter assembly for maintaining sterility of the intermittent catheter. The catheter housing includes a bottle and a pull tab. The bottle includes a neck. An entirety of the catheter tube is disposed in the bottle with the funnel fitted into the neck in the packaged state of the intermittent-catheter assembly. The pull tab seals the intermittent catheter in the bottle in the packaged state of the intermittent-catheter assembly.

In some embodiments, a proximal opening of the funnel and a proximal opening of the bottle are concentric. Being concentric, the pull tab simultaneously seals the funnel and the bottle in the packaged state of the intermittent-catheter assembly.

In some embodiments, the bottle is configured to be fluidly coupled to the funnel for voiding urine into the bottle upon catheterization with the intermittent catheter.

Also disclosed herein is a method of an intermittent-catheter assembly including, in some embodiments, a catheter assembly-obtaining step, an intermittent catheter-exposing step, an intermittent catheter-removing step, a catheter tube-inserting step, and a urine-voiding step. The catheter assembly-obtaining step includes obtaining the intermittent-catheter assembly in a packaged state of the intermittent-catheter assembly. The intermittent-catheter assembly has an intermittent catheter disposed in a catheter housing in the packaged state of the intermittent-catheter assembly. The intermittent catheter-exposing step includes exposing the intermittent catheter for removal from the catheter housing, which includes an outer sleeve-grasping step, a force-applying step, and an outer sleeve-sliding step. The outer sleeve-grasping step includes grasping an outer sleeve of the catheter housing. The force-applying step includes applying a force to an exposed end of an inner sleeve of the catheter housing. The outer sleeve-sliding step includes sliding the outer sleeve toward the exposed end of the inner sleeve in opposition to the force applied to the exposed end of the inner sleeve. The intermittent catheter-removing step includes removing the intermittent catheter from the catheter housing. The catheter tube-inserting step includes inserting a catheter tube of the intermittent catheter into a urethra. The urine-voiding step includes voiding urine from a bladder.

In some embodiments, the method further includes a catheter tube-removing step and a catheter assembly-disposing step. The catheter tube-removing step includes removing the catheter tube from the urethra after the urine-voiding step. The catheter assembly-disposing step includes disposing of the intermittent catheter and the catheter housing. Optionally, the intermittent catheter and the catheter housing are disposed of in a reassembled state or partially reassembled state of the intermittent-catheter assembly during the catheter assembly-disposing step.

Also disclosed herein is a method of an intermittent-catheter assembly including, in some embodiments, a catheter assembly-obtaining step, an intermittent catheter-exposing step, a catheter tube-inserting step, and a urine-voiding step. The catheter assembly-obtaining step includes obtaining the intermittent-catheter assembly in a packaged state of the intermittent-catheter assembly. The intermittent-catheter assembly has an intermittent catheter disposed in a catheter housing in the packaged state of the intermittent-catheter assembly. The intermittent catheter-exposing step includes exposing the intermittent catheter for insertion into a urethra, which includes a pull tab-removing step, an end piece-grasping step, and an end piece-sliding step. The pull tab-removing step includes removing a pull tab sealing a distal opening of an end piece of the catheter housing. The end piece-grasping step includes grasping the end piece. The end piece-sliding step includes proximally sliding the end piece over the catheter tube toward a funnel of the intermittent catheter. The catheter tube-inserting step includes inserting the catheter tube of the intermittent catheter into the urethra. The urine-voiding step includes voiding urine from a bladder.

In some embodiments, the method further includes a catheter tube-removing step and a catheter assembly-disposing step. The catheter tube-removing step includes removing the catheter tube from the urethra after the urine-voiding step. The catheter assembly-disposing step includes disposing of the intermittent catheter and the catheter housing. Optionally, the intermittent catheter and the catheter housing are disposed of in a reassembled state or partially reassembled state of the intermittent-catheter assembly during the catheter assembly-disposing step.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1A illustrates a first intermittent-catheter assembly in a packaged state of the intermittent-catheter assembly in accordance with some embodiments.

FIG. 1B illustrates the first intermittent-catheter assembly when an outer sleeve of a catheter housing is slid toward an exposed end of an inner sleeve of the catheter housing in accordance with some embodiments.

FIG. 1C illustrates the first intermittent-catheter assembly with an intermittent catheter ready to be removed from the catheter housing in accordance with some embodiments.

FIG. 2A illustrates a cross section of the first intermittent-catheter assembly of FIG. 1A in accordance with some embodiments.

FIG. 2B illustrates a cross section of the first intermittent-catheter assembly of FIG. 1B in accordance with some embodiments.

FIG. 2C illustrates a cross section of the first intermittent-catheter assembly of FIG. 1C in accordance with some embodiments.

FIG. 3 illustrates a longitudinal cross section of the catheter housing in accordance with some embodiments.

FIG. 4 illustrates a perspective view of the catheter housing in accordance with some embodiments.

FIG. 5A illustrates a second intermittent-catheter assembly in a packaged state of the intermittent-catheter assembly in accordance with some embodiments.

FIG. 5B illustrates the second intermittent-catheter assembly with an intermittent catheter ready to be removed from a catheter housing in accordance with some embodiments.

FIG. 5C illustrates the second intermittent-catheter assembly with the intermittent catheter and a drainage bag ready to be removed from the catheter housing in accordance with some embodiments.

FIG. 6A illustrates a tether of the second intermittent-catheter assembly in accordance with some embodiments.

FIG. 6B illustrates the tether bending away from a centerline of the catheter housing in accordance with some embodiments.

FIG. 7A illustrates a cross section of a third intermittent-catheter assembly in a packaged state of the intermittent-catheter assembly in accordance with some embodiments.

FIG. 7B illustrates a cross section of the third intermittent-catheter assembly with an intermittent catheter ready to be removed from a catheter housing in accordance with some embodiments.

FIG. 8 illustrates a detailed view of the third intermittent-catheter assembly with the intermittent catheter ready to be removed from the catheter housing in accordance with some embodiments.

FIG. 9 illustrates the third intermittent-catheter assembly with a first form factor in accordance with some embodiments.

FIG. 10 illustrates the third intermittent-catheter assembly with a second form factor in accordance with some embodiments.

FIG. 11 illustrates the third intermittent-catheter assembly with a third form factor in accordance with some embodiments.

FIG. 12 illustrates the third intermittent-catheter assembly with a fourth form factor in accordance with some embodiments.

FIG. 13A illustrates a fourth intermittent-catheter assembly in a packaged state of the intermittent-catheter assembly in accordance with some embodiments.

FIG. 13B illustrates the fourth intermittent-catheter assembly when an outer sleeve of a catheter housing is slid toward an exposed end of an inner sleeve of the catheter housing in accordance with some embodiments.

FIG. 13C illustrates the fourth intermittent-catheter assembly with an intermittent catheter and drainage bag ready to be removed from the catheter housing in accordance with some embodiments.

FIG. 14 illustrates the outer sleeve and the inner sleeve of the catheter housing separated from each other in accordance with some embodiments.

FIG. 15A illustrates a connecting portion of the inner sleeve disposed in the catheter housing in accordance with some embodiments.

FIG. 15B illustrates the connecting portion of the inner sleeve bending away from a centerline of the catheter housing in accordance with some embodiments.

FIG. 15C illustrates the connecting portion of the inner sleeve further bending away from the centerline of the catheter housing in accordance with some embodiments.

FIG. 16A illustrates a fifth intermittent-catheter assembly in a packaged state of the intermittent-catheter assembly in accordance with some embodiments.

FIG. 16B illustrates the fifth intermittent-catheter assembly with an intermittent catheter ready to be removed from a catheter housing in accordance with some embodiments.

FIG. 16C illustrates the fifth intermittent-catheter assembly with the intermittent catheter and a drainage bag ready to be removed from the catheter housing in accordance with some embodiments.

FIG. 17 illustrates a cap including a pull tab in accordance with some embodiments.

FIG. 18 illustrates a cap including a push tab in accordance with some embodiments.

FIG. 19 illustrates a cap including a push button in accordance with some embodiments.

FIG. 20 illustrates a sixth intermittent-catheter assembly in a packaged state of the intermittent-catheter assembly in accordance with some embodiments.

FIG. 21 illustrates a sleeve, a cap, and shrink-wrap packaging of the intermittent-catheter assembly of FIG. 20 separated from each other in accordance with some embodiments.

FIG. 22 illustrates the sixth intermittent-catheter assembly including a drainage bag in a packaged state of the intermittent-catheter assembly in accordance with some embodiments.

FIG. 23 illustrates a sleeve, a cap, and shrink-wrap packaging of the intermittent-catheter assembly of FIG. 22 separated from each other in accordance with some embodiments.

FIG. 24 illustrates a first cap including internal threads for screwing the cap off the sleeve in accordance with some embodiments.

FIG. 25 illustrates a second cap including internal threads for screwing the cap off the sleeve in accordance with some embodiments.

FIG. 26 illustrates a third cap configured for tearing the cap away from the sleeve in accordance with some embodiments.

FIG. 27A illustrates a seventh intermittent-catheter assembly in a packaged state of the intermittent-catheter assembly in accordance with some embodiments.

FIG. 27B illustrates the seventh intermittent-catheter assembly with an intermittent catheter ready to be removed from a catheter housing in accordance with some embodiments.

FIG. 28 illustrates a sleeve, a reinforcing insert, and an adhesive tab separated from each other in accordance with some embodiments.

FIG. 29A illustrates a seventh intermittent-catheter assembly including a drainage bag in a packaged state of the intermittent-catheter assembly in accordance with some embodiments.

FIG. 29B illustrates the seventh intermittent-catheter assembly with the intermittent catheter and the drainage bag ready to be removed from a catheter housing in accordance with some embodiments.

FIG. 30 illustrates the sleeve, the reinforcing insert, and the adhesive tab of the intermittent-catheter assembly of FIGS. 29A and 29B separated from each other in accordance with some embodiments.

FIG. 31A illustrates an eighth intermittent-catheter assembly in a packaged state of the intermittent-catheter assembly in accordance with some embodiments.

FIG. 31B illustrates the eighth intermittent-catheter assembly with an intermittent catheter ready to be inserted into a urethra in accordance with some embodiments.

FIG. 32A illustrates the eighth intermittent-catheter assembly including a drainage bag in a packaged state of the intermittent-catheter assembly in accordance with some embodiments.

FIG. 32B illustrates the eighth intermittent-catheter assembly of FIG. 32A with the intermittent catheter ready to be inserted into a urethra in accordance with some embodiments.

FIG. 33 illustrates a ninth intermittent-catheter assembly in a packaged state of the intermittent-catheter assembly in accordance with some embodiments.

FIG. 34 illustrates a tenth intermittent-catheter assembly in a packaged state of the intermittent-catheter assembly in accordance with some embodiments.

FIG. 35 illustrates a bottom suction cup for an intermittent-catheter assembly in accordance with some embodiments.

FIG. 36 illustrates a side suction cup for an intermittent-catheter assembly in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, users of urinary catheters such as intermittent catheters self-catheterize four to six times a day. As such, a simple-to-use intermittent catheter that ensures sterility before use and facilitates cleanliness after use is needed.

Disclosed herein are intermittent-catheter assemblies and methods thereof that address the foregoing.

The intermittent-catheter assemblies set forth below include an intermittent catheter and a catheter housing. The intermittent catheter can generally be used in any of the intermittent-catheter assemblies. As such, the intermittent catheter is described first followed by a number of different intermittent-catheter assemblies, which assemblies largely focus on the catheter housings thereof. Should the intermittent catheter need any modifications for use with a particular intermittent-catheter assembly, such modifications are described in relation to the particular intermittent-catheter assembly if useful for understanding the particular intermittent-catheter assembly. Methods of the intermittent-catheter assemblies are described after the intermittent-catheter assemblies.

### Intermittent Catheters

As shown in any figure of a number of figures, an intermittent catheter 10 (e.g., a female intermittent catheter) includes a funnel 12 and a catheter tube 14 fluidly coupled to the funnel 12.

The funnel 12 is configured to provide a handle for holding the intermittent catheter 10 while removing the intermittent catheter 10 from a catheter housing or voiding urine through a proximal opening 18 of the funnel 12. The funnel 12 can include a neck 16 into which the catheter tube 14 is inserted. If the intermittent catheter 10 does not include the neck 16, the catheter tube 14 is inserted into a distal opening of the funnel 12. The funnel 12, the neck 16, or both the funnel 12 and the neck 16 can include a plurality of ridges integrated into an outer surface thereof. The ridges can be circumferential ridges configured for gripping the funnel 12 or the neck 16 as a handle while removing the intermittent catheter from the catheter housing or voiding the urine.

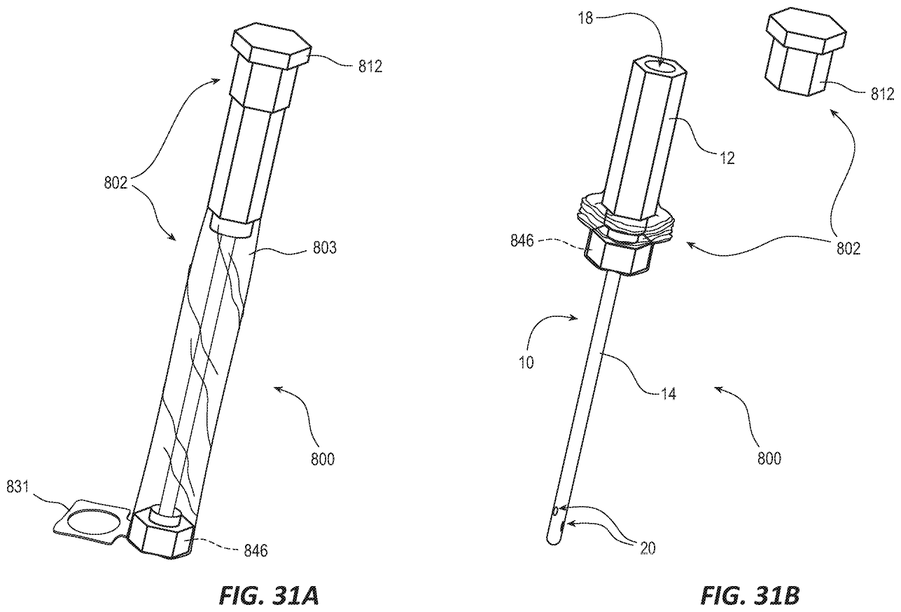

The catheter tube 14 is configured for insertion into a urethra for voiding urine from a bladder. The catheter tube 14 includes one or more eyelets 20 proximate a catheter tip. (See FIGS. 31B, 32B, 33, 34) The one-or-more eyelets 20 are in fluid communication with the proximal opening 18 of the funnel 12 by way of a catheter-tube lumen extending along a length of the catheter tube 14.

The intermittent catheter 10 can further include a lubricant disposed on the catheter tube 14 such as coating the catheter tube 14.

### Intermittent Catheter Assemblies

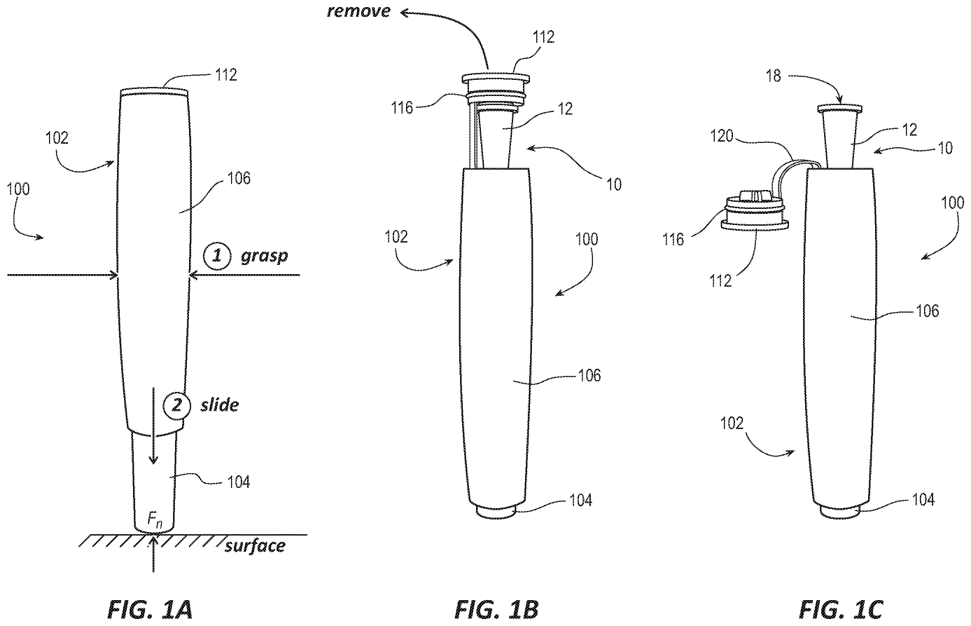

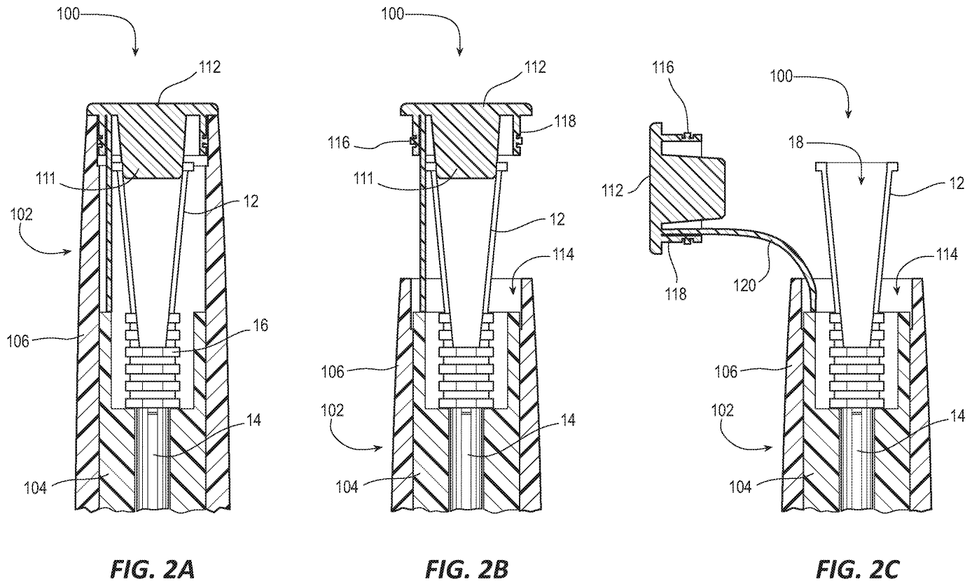

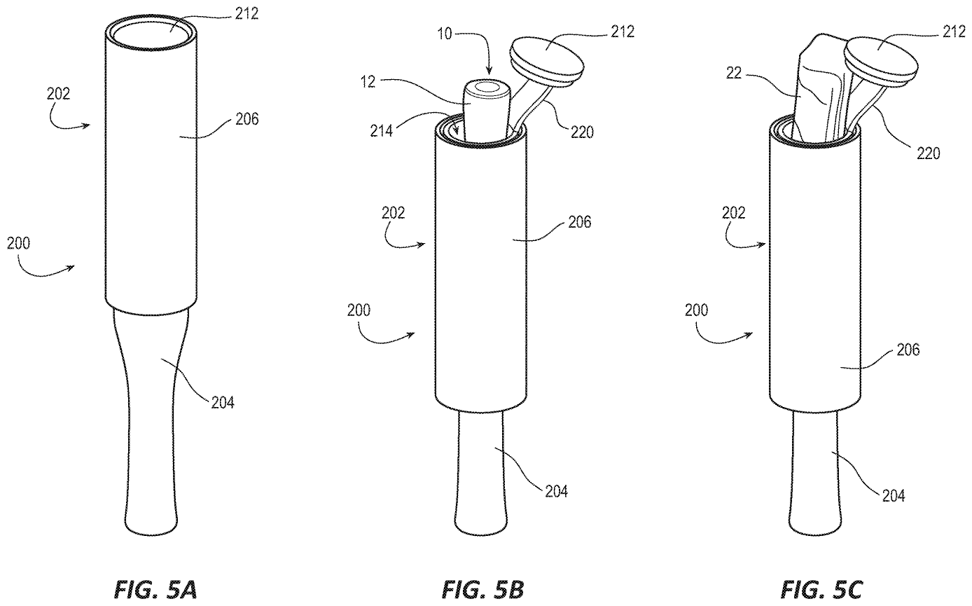

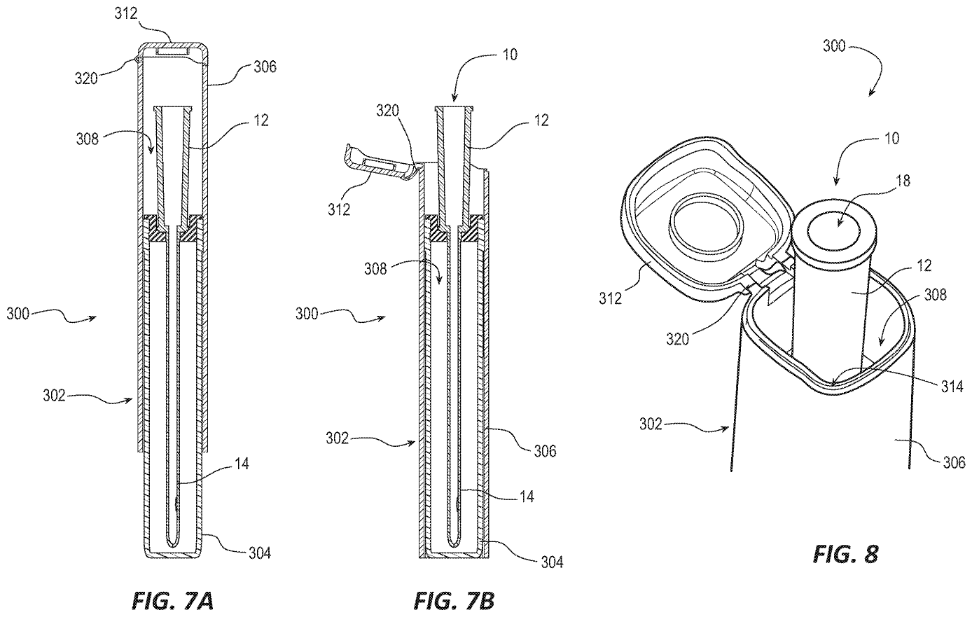

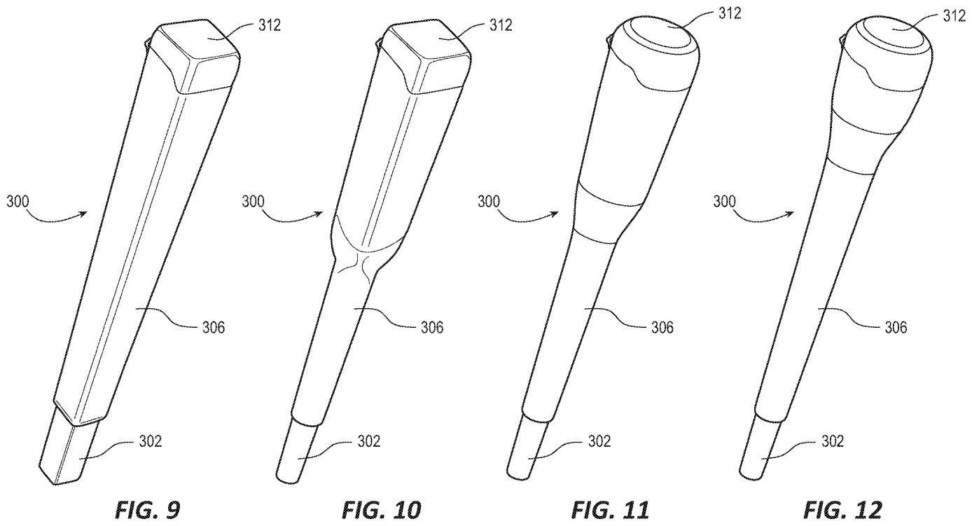

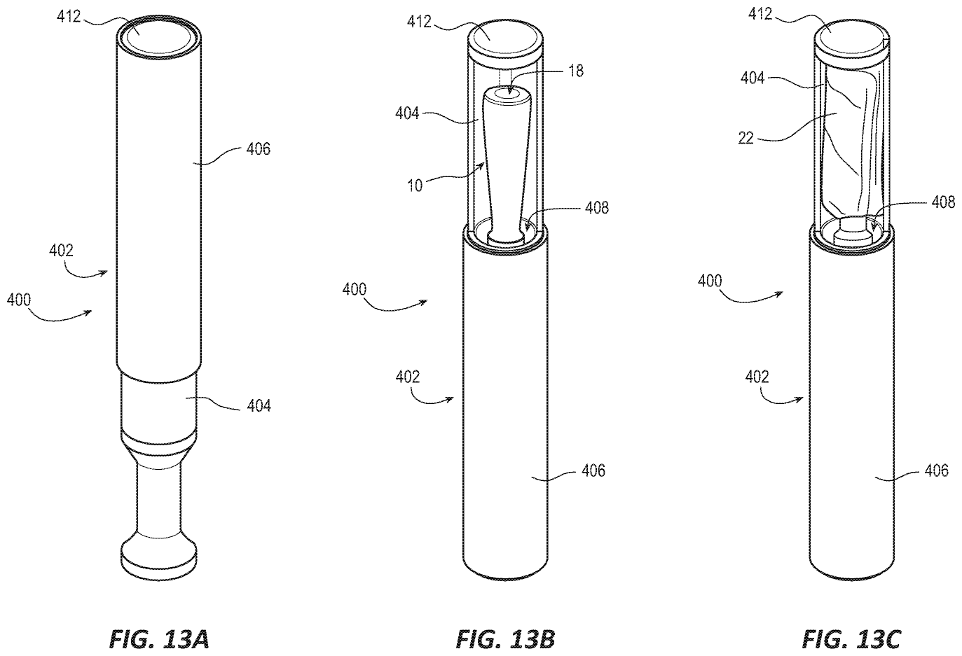

FIGS. 1A-1C and 2A-2C illustrate different views of a first intermittent-catheter assembly 100 in different states in accordance with some embodiments. FIGS. 5A-5C illustrate a second intermittent-catheter assembly 200 in different states in accordance with some embodiments. FIGS. 7A, 7B, and 8 illustrate a third intermittent-catheter assembly 300 in different states in accordance with some embodiments. In consideration of the intermittent-catheter assembly 300 of FIGS. 7A, 7B, and 8 having a first form factor, FIGS. 9-12 illustrate second, third, fourth, and fifth form factors for the intermittent-catheter assembly 300 in accordance with some embodiments. FIGS. 13A-13C illustrate a fourth intermittent-catheter assembly 400 in different states in accordance with some embodiments.

As shown, the intermittent-catheter assembly 100, 200, 300, or 400 includes the intermittent catheter 10 and a catheter housing 102, 202, 302, or 402. The intermittent catheter 10 is disposed in the catheter housing 102, 202, 302, or 402 while in a packaged state of the intermittent-catheter assembly 100, 200, 300, or 400 for maintaining sterility of the intermittent catheter 10.

The catheter housing 102, 202, 302, or 402 includes an inner sleeve 104, 204, 304, or 404 and an outer sleeve 106, 206, 306, or 406. The inner sleeve 104, 204, 304, or 404 includes a longitudinal cavity 108, 208, 308, or 408 containing a majority of the intermittent catheter 10 in the packaged state of the intermittent-catheter assembly 100, 200, 300, or 400. The outer sleeve 106, 206, 306, or 406 is slidably disposed over the inner sleeve 104, 204, 304, or 404. The catheter housing 102, 202, 302, or 402 is configured to expose the intermittent catheter 10 for removal from the catheter housing 102, 202, 302, or 402 when the outer sleeve 106, 206, 306, or 406 is grasped and slid toward an exposed end of the inner sleeve 104, 204, 304, or 404 in opposition to a force (e.g., a normal force Fₙ) applied to the exposed end of the inner sleeve 104, 204, 304, or 404 such as by holding the exposed end of the inner sleeve 104, 204, 304, or 404 against a surface (e.g., a tabletop, a palm of a hand, etc.).

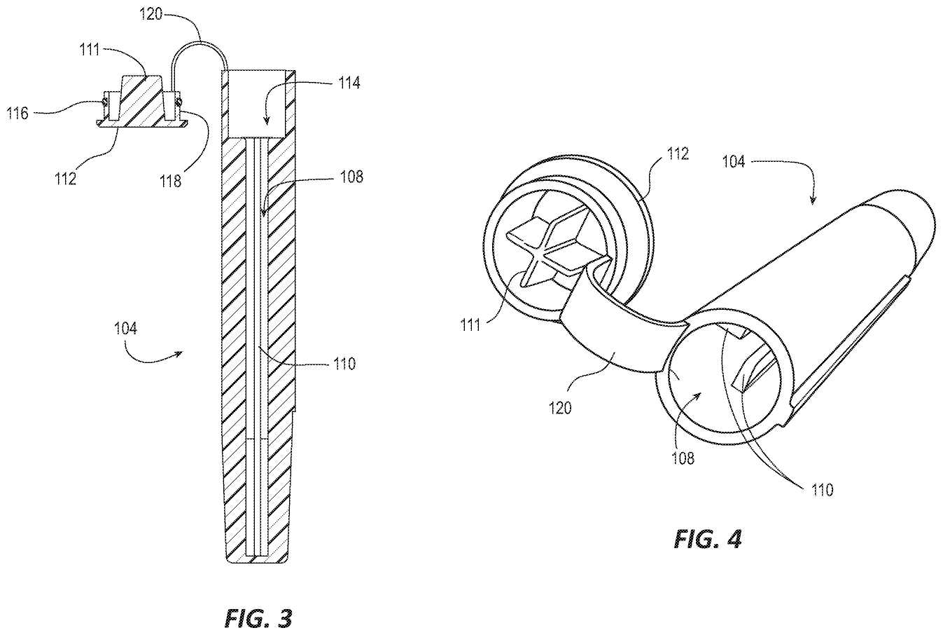

FIG. 3 illustrates a longitudinal cross section of the catheter housing 102 in accordance with some embodiments. FIG. 4 illustrates a perspective view of the catheter housing 102 in accordance with some embodiments.

As shown by the longitudinal cross section of the catheter housing 102, the inner sleeve 104, 204, 304, or 404 can include longitudinal ribs 110 such as those shown for the inner sleeve 104 extending into the cavity 108. When present, the ribs 110 stabilize the intermittent catheter 10 in the inner sleeve 104, 204, 304, or 404 in the packaged state of the intermittent-catheter assembly 100, 200, 300, or 400.

In addition to the ribs 110, at least the cap 112 or 212 set forth below can include a funnel insert 111 along a centerline of the cap 112 or 212 as shown in FIGS. 3 and 4 for the catheter housing 102. Indeed, the funnel insert 111 is shown inboard of the rim 118 of the cap 112. When present, the funnel insert 111 is inserted into the funnel 12 of the intermittent catheter 10 in the packaged state of the intermittent-catheter assembly 100 or 200, which stabilizes the intermittent catheter 10 in the inner sleeve 104 or 204.

To reduce redundancy in description set forth herein, the ribs 110, the funnel insert 111, or both the ribs 110 and the funnel insert 111 can be incorporated into any intermittent-catheter assembly of those set forth herein to the same effect as the foregoing. This is with the understanding that certain modifications within the skill of a person of ordinary skill in the art might need to be made to the intermittent-catheter assembly to which the ribs 110 or the funnel insert 111 is added.

Adverting to the intermittent-catheter assemblies 100 and 200, the inner sleeve 104 or 204 includes a displaceable cap 112 or 212 sealing an opening of the outer sleeve 106 or 206 opposite the exposed end of the inner sleeve 104 or 204 in the packaged state of the intermittent-catheter assembly 100 or 200. Indeed, the cap 112 or 212 seals the opening of the outer sleeve 106 or 206 and maintains sterility of the intermittent catheter 10 in the packaged state of the intermittent-catheter assembly 100 or 200.

The cap 112 or 212 sits in a seat 114 or 214 formed within the opening of the outer sleeve 106 or 206. As shown for the catheter housing 102, the cap 112 or 212 can include an annular gasket 116 such as that disposed in a recess around a rim 118 of the cap 112. When present, the gasket 116 sits between the cap 112 or 212 and the outer sleeve 106 or 206 securing and sealing the cap 112 or 212 in the outer sleeve 106 or 206 in the packaged state of the intermittent-catheter assembly 100 or 200.

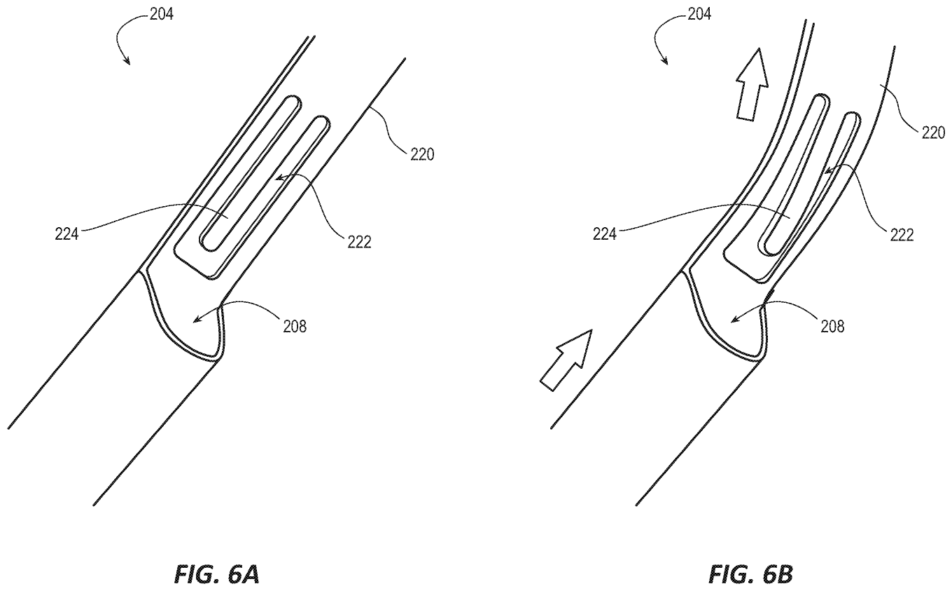

FIG. 6A illustrates a tether 220 of the catheter housing 202 in accordance with some embodiments. FIG. 6B illustrates the tether 220 bending away from a centerline of the catheter housing 202 in accordance with some embodiments.

As shown in FIGS. 1C, 2C, 3, and 4 for the catheter housing 102 and FIGS. 5B, 5C, 6A, and 6B for the catheter housing 202, the cap 112 or 212 can be tethered to the inner sleeve 104 or 204 by the tether 120 or 220 to keep the cap 112 or 212 with the catheter housing 102 or 202. Upon grasping the outer sleeve 106 or 206 and sliding the outer sleeve 106 or 206 toward the exposed end of the inner sleeve 104 or 204 in opposition to a force (e.g., a normal force Fₙ) applied to the exposed end of the inner sleeve 104 or 204, the cap 112 or 212 pops open to expose the funnel 12 of the intermittent catheter 10 but remains attached to the inner sleeve 104 or 204 by the tether 120 or 220.

The tether 120 or 220 can be a polymeric wire or ribbon. As to the tether 220, the tether 220 is shown in FIGS. 6A and 6B as a ribbon with an opening 222 configured to weaken a structural integrity of the tether 220 around the opening 222 such that the tether 220 and the cap 212 tethered thereto bend away from a centerline of the catheter housing 202 as the outer sleeve 206 is slid toward the exposed end of the inner sleeve 204. So configured, the tether 220 facilitates access to the intermittent catheter 10 once exposed. In addition, the opening 222 can include a tongue 224 in about a same plane or surface as the opening 222, which tongue can be used like a belt clip to suspend the intermittent-catheter assembly 200. Likewise, the tether 120 can include such an opening an optional tongue.

Adverting to the intermittent-catheter assembly 300, the outer sleeve 306 includes a displaceable cap 312 sealing an opening of the outer sleeve 306 opposite the exposed end of the inner sleeve 304 in the packaged state of the intermittent-catheter assembly 300. Indeed, the cap 312 seals the opening of the outer sleeve 306 and maintains sterility of the intermittent catheter 10 in the packaged state of the intermittent-catheter assembly 300.

The cap 312 sits on a seat 314 formed around the opening of the outer sleeve 306 in the packaged state of the intermittent-catheter assembly 300. As shown, the cap 312 and the outer sleeve 306 can include complementary snap-fit features. When present, the complementary snap-fit features secure the cap 312 on the outer sleeve 306 in the packaged state of the intermittent-catheter assembly 300.

As best shown in FIG. 8, the cap 312 can be coupled to the outer sleeve 306 by a living hinge 320 to keep the cap 312 with the catheter housing 302. Upon grasping the outer sleeve 306 and sliding the outer sleeve 306 toward the exposed end of the inner sleeve 304 in opposition to a force (e.g., a normal force Fₙ) applied to the exposed end of the inner sleeve 304, the cap 312 pops open to expose the funnel 12 of the intermittent catheter 10 but remains attached to the outer sleeve 306 by the living hinge 320.

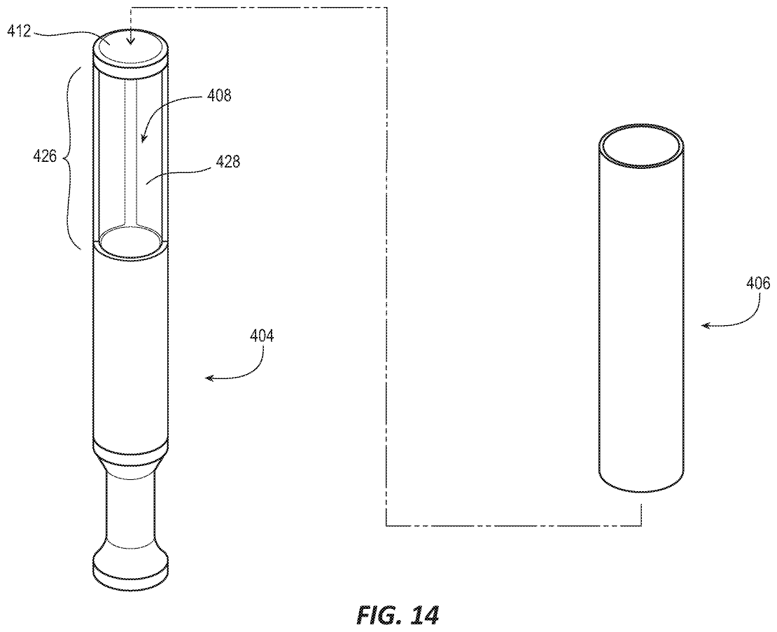

FIG. 14 illustrates the inner sleeve 404 and the outer sleeve 406 of the catheter housing 402 separated from each other in accordance with some embodiments.

Adverting to the intermittent-catheter assembly 400, the inner sleeve 404 includes an end cap 412 integrated into the inner sleeve 404. Indeed, the inner sleeve 404 includes a longitudinal side opening 426 to the longitudinal cavity 408 with the end cap 412 integrated into the inner sleeve 404 on an end of the side opening 426 opposite the exposed end of the inner sleeve 404 in the packaged state of the intermittent-catheter assembly 300.

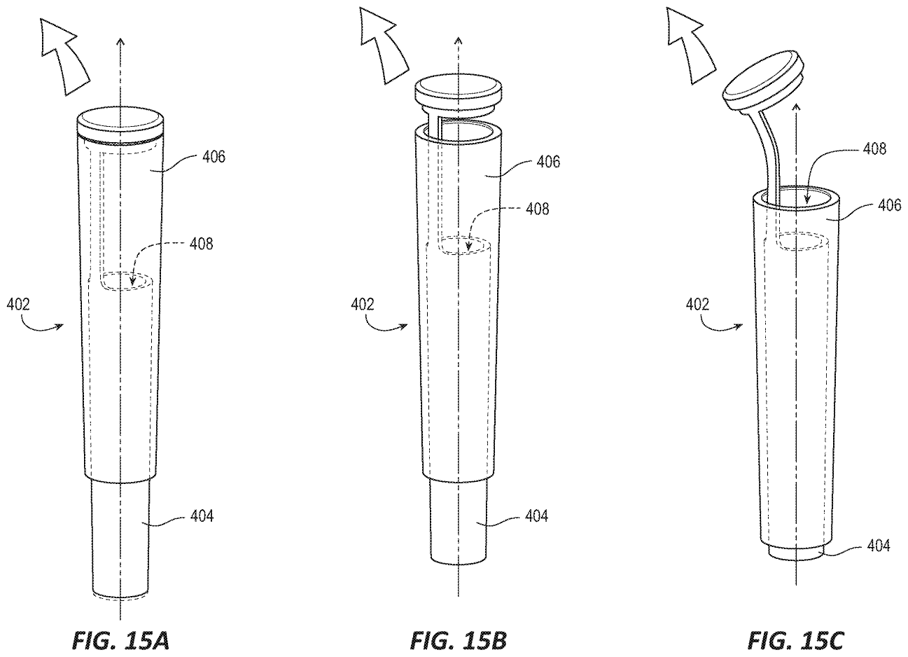

FIG. 15A illustrates a connecting portion 428 of the inner sleeve 404 disposed in the catheter housing 402 in accordance with some embodiments, and FIGS. 15B and 15C illustrate the connecting portion 428 of the inner sleeve bending away from a centerline of the catheter housing 402 in accordance with some embodiments.

As shown, the connecting portion 428 of the inner sleeve 404 is coterminous with ends of the side opening 426. The connecting portion 428 is molded with a bias such that the connecting portion 428 and the end cap 412 coupled thereto bend away from the centerline of the catheter housing 402 as the outer sleeve 406 is slid toward the exposed end of the inner sleeve 404. So configured, the connecting portion 428 facilitates access to the intermittent catheter 10. Indeed, upon grasping the outer sleeve 406 and sliding the outer sleeve 406 toward the exposed end of the inner sleeve 404 in opposition to a force (e.g., a normal force Fₙ) applied to the exposed end of the inner sleeve 404, the connecting portion 428 bends away from the centerline of the catheter housing 402 and the funnel 12 of the intermittent catheter 10.

As shown in FIGS. 5C and 13C by the intermittent-catheter assemblies 200 and 400, the intermittent-catheter assembly 100, 200, 300, or 400 can include a drainage bag 22. The drainage bag 22 is fluidly coupled to the funnel 12 for voiding urine into the drainage bag 22 upon catheterization with the intermittent catheter 10.

To reduce redundancy in description set forth herein, the drainage bag can be incorporated into any intermittent-catheter assembly of those set forth herein to the same effect as the foregoing. This is with the understanding that certain modifications within the skill of a person of ordinary skill in the art might need to be made to the intermittent-catheter assembly to which the drainage bag 22 is added.

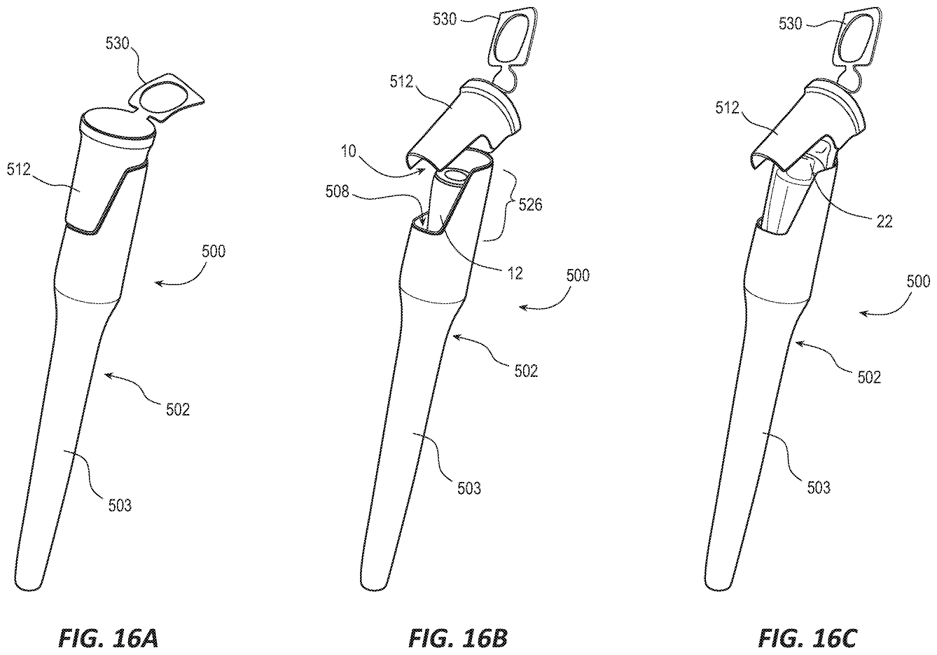

FIGS. 16A-16C illustrate a fifth intermittent-catheter assembly 500 in different states in accordance with some embodiments.

As shown, the intermittent-catheter assembly 500 includes the intermittent catheter 10 and a catheter housing 502. The intermittent catheter 10 is disposed in the catheter housing 502 while in a packaged state of the intermittent-catheter assembly 500 for maintaining sterility of the intermittent catheter 10.

The catheter housing 502 includes a sleeve 503 and a cap 512 for sealing an opening of the sleeve 503 opposite a closed end of the sleeve 503. Indeed, the cap 512 seals the opening of the sleeve 503 and maintains sterility of the intermittent catheter 10 in the packaged state of the intermittent-catheter assembly 500.

The sleeve 503 includes a longitudinal cavity 508 containing a majority of the intermittent catheter 10 in the packaged state of the intermittent-catheter assembly 500. The cavity 508 is accessible by way of a longitudinal extension 526 of the opening into the sleeve 503. The extension 526 into the sleeve 503 is configured to expose a longitudinal portion of the funnel 12 when the cap 512 is moved from a closed position in the packaged state of the intermittent-catheter assembly to an open position such as the open position of the cap 512 about the living hinge 520 set forth below.

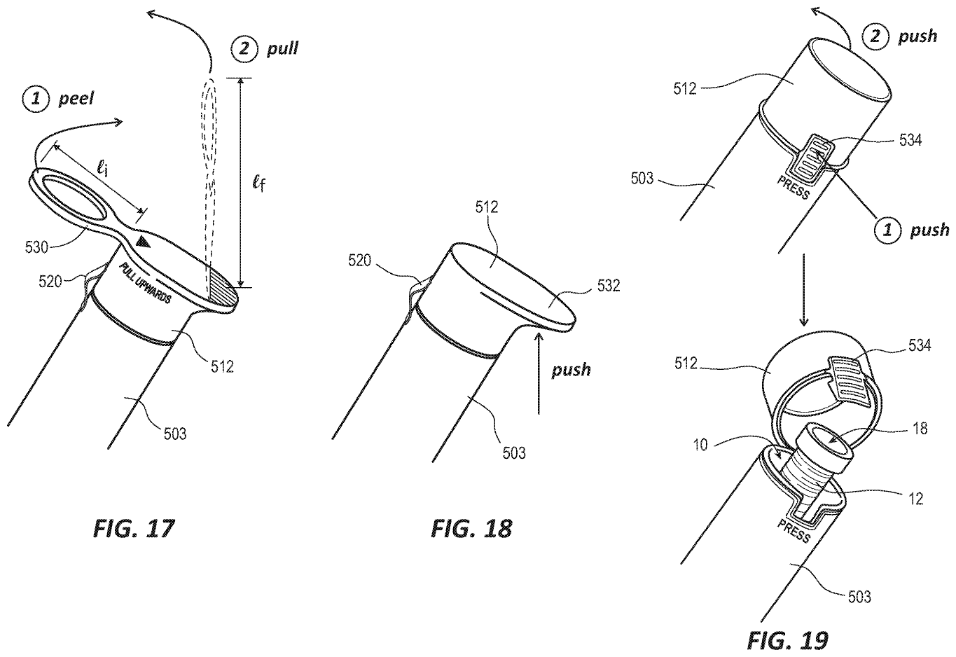

The cap 512 can be coupled to the sleeve 503 by a living hinge 520 to keep the cap 512 with the catheter housing 502. (See FIGS. 17 and 18.) As set forth below, the cap 512 can alternatively be any cap of the those set forth in FIGS. 16A-16C and 17-19, which might require certain modifications to the sleeve 503 as well.

FIG. 17 illustrates the cap 512 including a pull tab 530 in accordance with some embodiments. In consideration of the cap 512 of FIGS. 16A and 16B having a first form factor, FIG. 17 illustrates a second form factor for the cap 512 of the intermittent-catheter assembly 500 in accordance with some embodiments. Indeed, the second form factor of the cap 512 does not extend into a longitudinal extension of the opening of the sleeve 503. That, and the second form factor of the cap 512 develops a longer lever arm than the first form factor of the cap 512 when the pull tab 530 is pulled away from the cap 512 as set forth below.

As shown, the cap 512 of FIGS. 16A, 16B, and 17 includes the pull tab 530 coupled to the cap 512 on a same side of the catheter housing 502 as the living hinge 520. A portion of the pull tab 530 is configured to peel away from the cap 512 toward an opposite side of the catheter housing 502 from the living hinge 520 when the pull tab 530 is initially pulled to extend a lever arm l with respect to the living hinge 520 from an initial lever arm lᵢ to a final lever arm l_{f} sufficient for subsequently pulling the cap 512 away from the opening of the sleeve 503 by the pull tab 530. Advantageously, when the pull tab 530 is intact (i.e., not pulled toward the opposite side of the catheter housing 502), the pull tab 530 indicates the intermittent-catheter assembly 500 has not been opened or tampered with by another party.

FIG. 18 illustrates the cap 512 including a push tab 532 in accordance with some embodiments.

As shown, the cap 512 of FIG. 18 includes the push tab 532 extending from an opposite side of the catheter housing 502 from the living hinge 520. The push tab 532 is configured to extend the lever arm l with respect to the living hinge 520 over that of the cap 512 without the push tab 532 such that the lever arm l is sufficient for pushing the cap 512 away from the opening of the sleeve 503 by the push tab 532.

FIG. 19 illustrates the cap 512 including a push button 534 in accordance with some embodiments.

As shown, the cap 512 of FIG. 19 includes the push button 534 on an opposite side of the catheter housing 502 from the living hinge 520. The push button 534 is configured to deform the cap 512 and disengage complementary snap-fit features between the cap 512 and the sleeve 503 for subsequently pushing the cap 512 away from the opening of the sleeve 503 by the push button 534.

To reduce redundancy in description set forth herein, the cap 512 set forth in any figure of FIGS. 16A-16C and 17-19 can be incorporated into any intermittent-catheter assembly of those set forth herein to the same effect as the foregoing. This is with the understanding that certain modifications within the skill of a person of ordinary skill in the art might need to be made to the intermittent-catheter assembly to which the cap 512 is added.

As shown in FIG. 16C, the intermittent-catheter assembly 500 can include the drainage bag 22. The drainage bag 22 is fluidly coupled to the funnel 12 for voiding urine into the drainage bag 22 upon catheterization with the intermittent catheter 10.

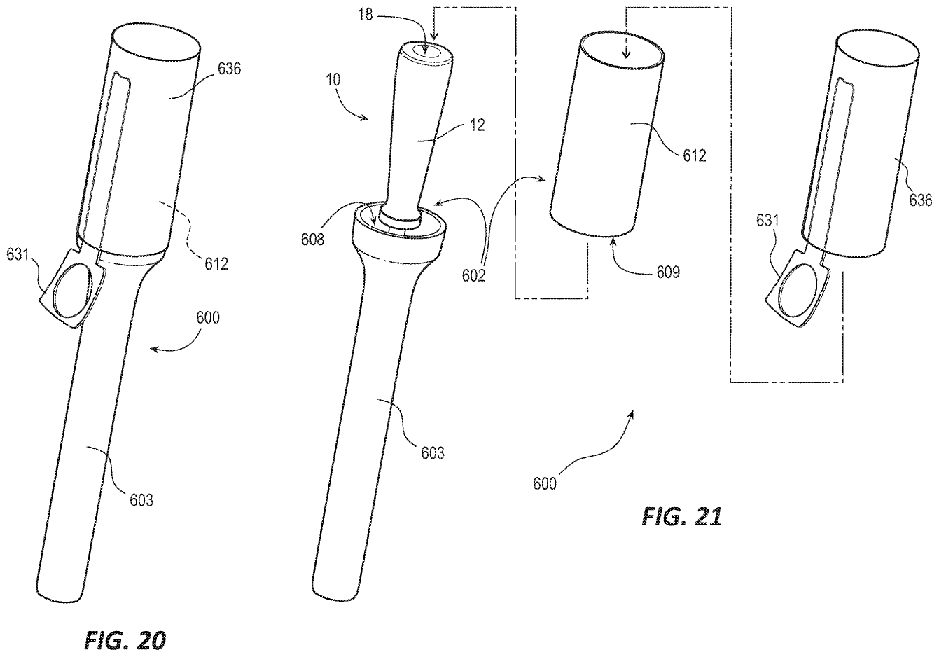

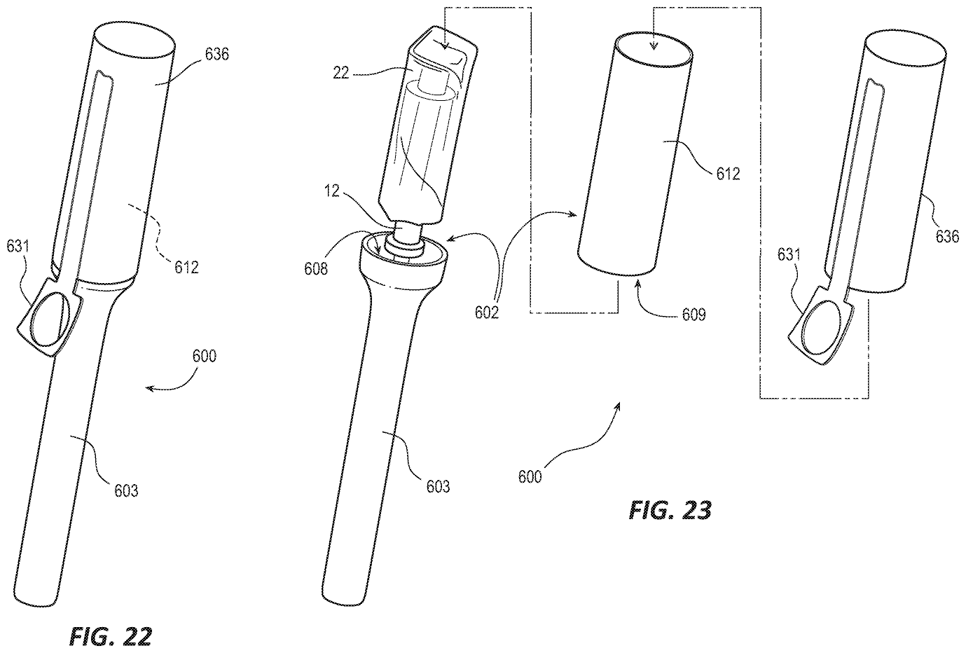

FIG. 20 illustrates a sixth intermittent-catheter assembly 600 in a packaged state of the intermittent-catheter assembly 600 in accordance with some embodiments. FIG. 21 illustrates a sleeve 603, a removable cap 612, and shrink-wrap packaging 636 of the intermittent-catheter assembly 600 of FIG. 20 separated from each other in accordance with some embodiments. FIGS. 22 and 23 illustrate the same intermittent-catheter assembly 600 albeit with an elongated form factor for inclusion of the drainage bag 22.

As shown, the intermittent-catheter assembly 600 includes the intermittent catheter 10 and a catheter housing 602. The intermittent catheter 10 is disposed in the catheter housing 602 while in the packaged state of the intermittent-catheter assembly 600 for maintaining sterility of the intermittent catheter 10.

The catheter housing 602 includes the sleeve 603 and the cap 612 for sealing an opening of the sleeve 603 opposite a closed end of the sleeve 603. Indeed, the cap 612 seals the opening of the sleeve 603 and maintains sterility of the intermittent catheter 10 in the packaged state of the intermittent-catheter assembly 600.

The sleeve 603 includes a longitudinal cavity 608 containing a majority of the intermittent catheter 10 in the packaged state of the intermittent-catheter assembly 600. The sleeve 603 including the cavity 608 thereof is approximately coextensive with the catheter tube 14 of the intermittent catheter 10.

While not shown, the sleeve 603 can include the ribs 110 extending into the cavity 108. When present, the ribs 110 stabilize the intermittent catheter 10 in the sleeve 603 in the packaged state of the intermittent-catheter assembly 600.

The cap 612 includes another longitudinal cavity 609 configured to contain a remainder of the intermittent catheter 10. The cap 612 including the cavity 609 thereof is approximately coextensive with the funnel 12 of the intermittent catheter 10.

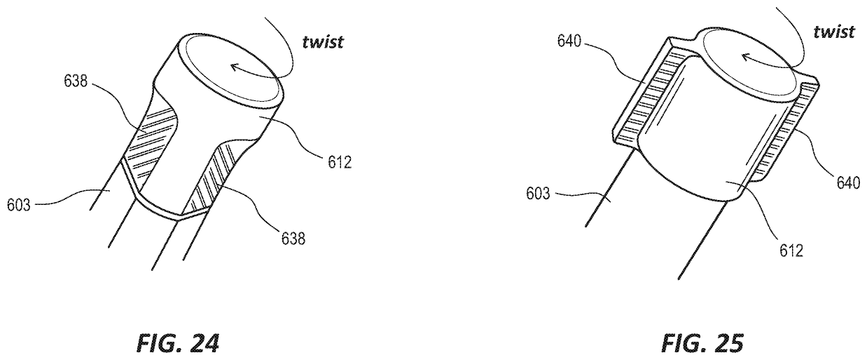

The sleeve 603 and the cap 612 can include complementary snap-fit features. When present, the complementary snap-fit features secure the cap 612 on the sleeve 603 in the packaged state of the intermittent-catheter assembly 600. In addition, the cap 612 can be configured to deform when squeezed. When the cap 612 is squeezed and deformed, the snap-fit features of the cap 612 disengage from those of the sleeve 603 for subsequently removing the cap 612. As set forth below, the cap 612 can be any cap of the those set forth in FIGS. 24-26, which might require certain modifications to the sleeve 603 as well.

FIG. 24 illustrates the cap 612 including longitudinal recesses 638 around an outside of the cap 612 in accordance with some embodiments. FIG. 25 illustrates the cap 612 including longitudinal ribs 640 around the outside of the cap 612 in accordance with some embodiments.

While not shown, the cap 612 of FIGS. 24 and 25 includes internal threads about an open-ended portion of the cap 612. In addition, the sleeve 603 includes complementary external threads about an open-ended portion of the sleeve 603 terminating with the opening of the sleeve 603. The recesses 638 or the ribs 640 around the outside of the cap 612 are configured to facilitate gripping the cap 612 and screwing the cap 612 off of the sleeve 603 or onto the sleeve 603.

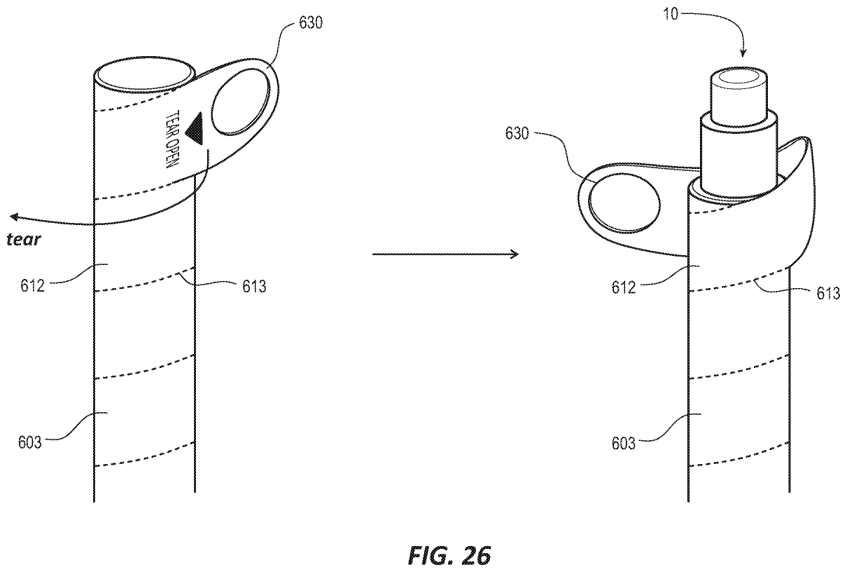

FIG. 26 illustrates the cap 612 including a pull tab 630 in accordance with some embodiments.

As shown, the cap 612 of FIG. 26 includes a circumferential weakened area 613 (e.g., a spiral perforation) around the cap 612, which can be a portion of the sleeve 603 opposite the closed end of the sleeve 603. The pull tab 630 is configured for gripping the cap 612 and tearing the cap 612 away from the sleeve 603. Advantageously, when the cap 612 is intact (i.e., not torn away from the sleeve 603), the cap 612 indicates the intermittent-catheter assembly 600 has not been opened or tampered with by another party.

To reduce redundancy in description set forth herein, the cap 612 set forth in any figure of FIGS. 20-26 can be incorporated into any intermittent-catheter assembly of those set forth herein to the same effect as the foregoing. This is with the understanding that certain modifications within the skill of a person of ordinary skill in the art might need to be made to the intermittent-catheter assembly to which the cap 612 is added.

The packaging 636 is over the entirety of the cap 612 and at least a portion of the sleeve 603 in at least the embodiments of the intermittent-catheter assembly 600 shown in FIGS. 20-23. The packaging 636 includes a pull tab 631 extending from the packaging 636 configured to break open the packaging 636 when the pull tab 631 is pulled.

As shown in FIG. 23, the intermittent-catheter assembly 600 can include the drainage bag 22. The drainage bag 22 is fluidly coupled to the funnel 12 for voiding urine into the drainage bag 22 upon catheterization with the intermittent catheter 10.

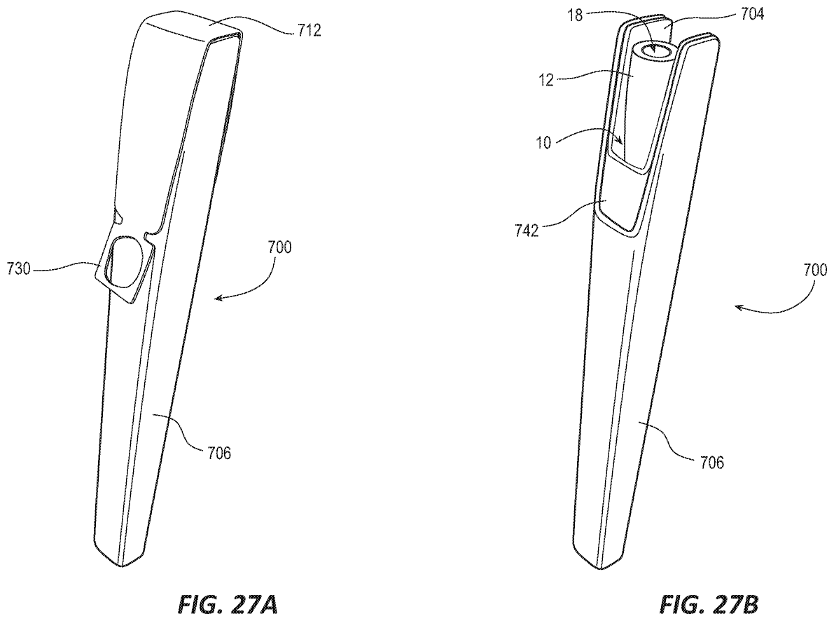

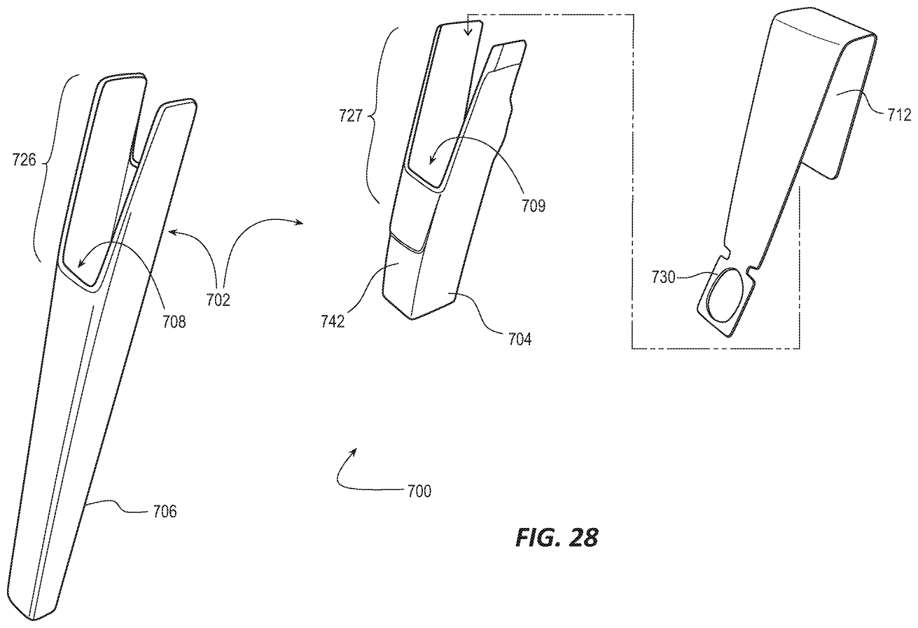

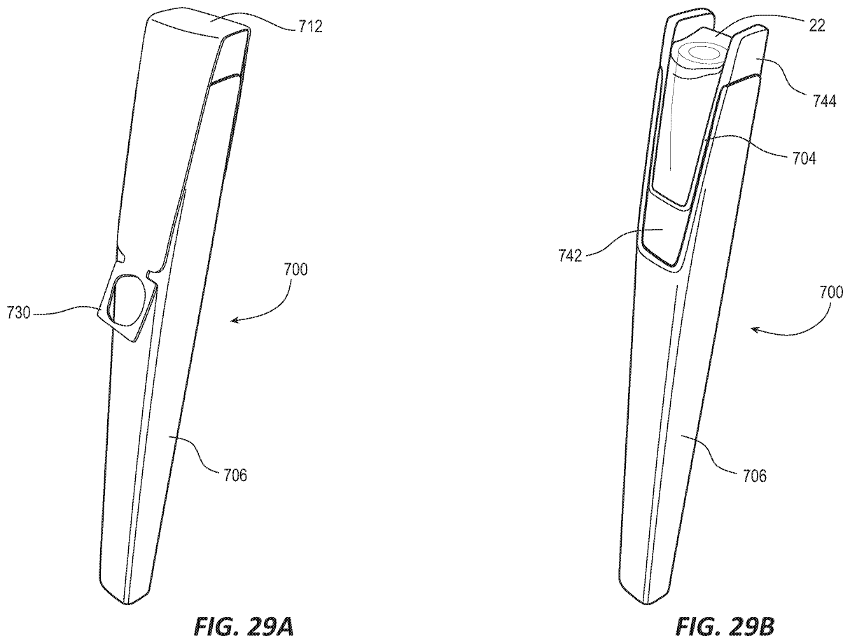

FIGS. 27A and 27B illustrate a seventh intermittent-catheter assembly 700 in different states of the intermittent-catheter assembly 700 in accordance with some embodiments. FIG. 28 illustrates a sleeve 706, a reinforcing insert 704, and an adhesive tab 712 of the intermittent-catheter assembly 700 of FIG. 27 A and 27B separated from each other in accordance with some embodiments. FIGS. 28A, 28B, and 29 illustrate the same intermittent-catheter assembly 700 albeit with an elongated form factor for inclusion of the drainage bag 22.

As shown, the intermittent-catheter assembly 700 includes the intermittent catheter 10 and a catheter housing 702. The intermittent catheter 10 is disposed in the catheter housing 702 while in a packaged state of the intermittent-catheter assembly 700 for maintaining sterility of the intermittent catheter.

The sleeve 706 includes a longitudinal cavity 708 containing a majority of the intermittent catheter 10 up to an entirety of the intermittent catheter 10 in the packaged state of the intermittent-catheter assembly 700. The sleeve 706 also includes a longitudinal sleeve gap 726 in major sides (e.g., a front and a back) of the sleeve 706 configured to provide access to the intermittent catheter 10 as set forth below.

The reinforcing insert 704 includes a longitudinal cavity 709 containing a portion (e.g., the funnel 12) of the intermittent catheter 10 in the packaged state of the intermittent-catheter assembly 700. The reinforcing insert 704 includes a longitudinal insert gap 727 and major-side protrusions 742, minor-side protrusions 744, or both the major-side protrusions 742 and the minor-side protrusions 744. The major-side and minor-side protrusions 742 and 744 are configured to suspend the reinforcing insert 704 in the sleeve gap 726 of the sleeve 706 such that the sleeve gap 726 and the insert gap 727 combine to provide major-side openings in the catheter housing 702 for grasping the funnel 12 and removing the intermittent catheter 10 from the catheter housing after the adhesive tab 712 is removed.

The adhesive tab 712 covers the major-side openings of the catheter housing 702 in the packaged state of the intermittent-catheter assembly 700. The adhesive tab 712 includes a pull tab 730 extending from the adhesive tab 712 configured to peel the adhesive tab 712 away from the major-side openings of the catheter housing 702 when the pull tab 730 is pulled.

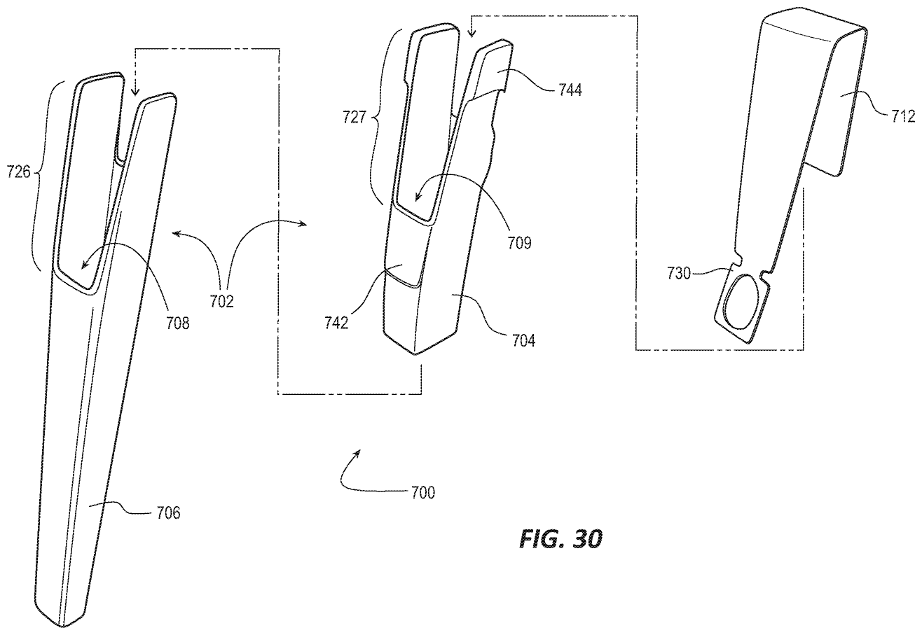

As shown in FIG. 29B, the intermittent-catheter assembly 700 can include the drainage bag 22. The drainage bag 22 is fluidly coupled to the funnel 12 for voiding urine into the drainage bag 22 upon catheterization with the intermittent catheter 10. Notably, the reinforcing insert 704 includes the minor-side protrusions 744 to lengthen the catheter housing 702 for accommodating the drainage bag 22 in the elongated form factor of the intermittent-catheter assembly 700 of FIGS. 29A, 29B, and 30.

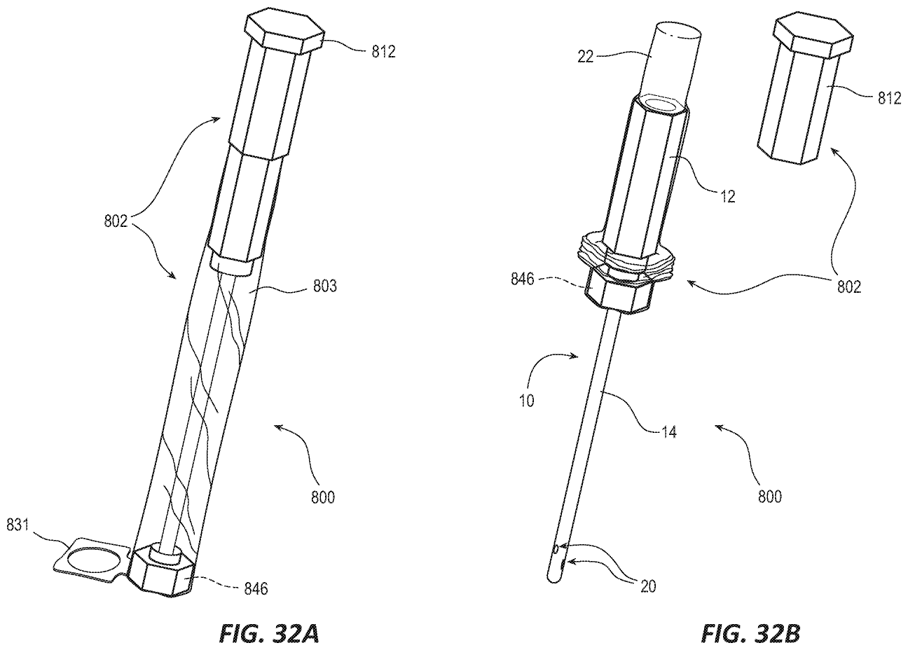

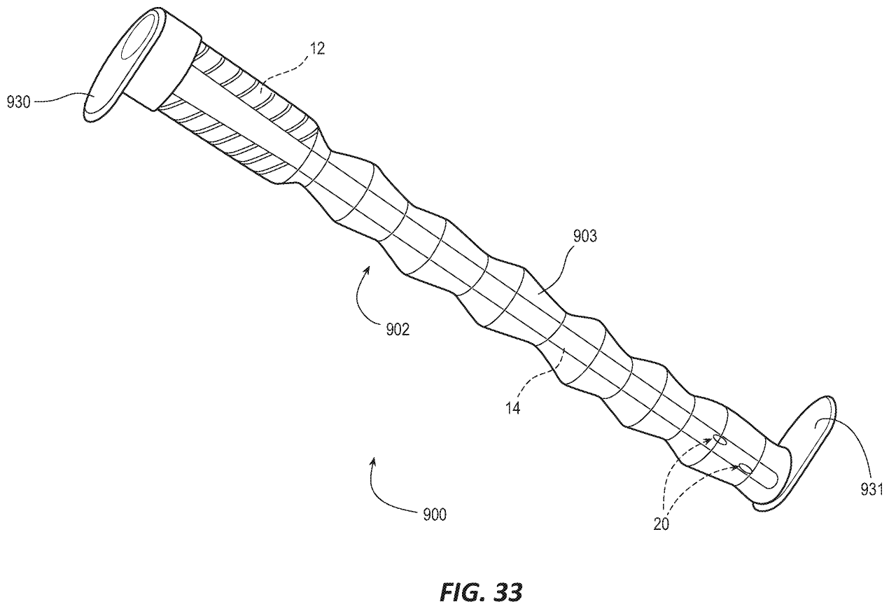

FIGS. 31A and 31B illustrate an eighth intermittent-catheter assembly 800 in different states of the intermittent-catheter assembly 800 in accordance with some embodiments. FIGS. 32A and 32B illustrate the same intermittent-catheter assembly 800 albeit with an elongated form factor for inclusion of the drainage bag 22. FIG. 33 illustrates a ninth intermittent-catheter assembly 900 in a packaged state of the intermittent-catheter assembly 900 in accordance with some embodiments.

As shown, the intermittent-catheter assembly 800 or 900 includes the intermittent catheter 10 and a catheter housing 802 or 902. The intermittent catheter 10 is disposed in the catheter housing 802 or 902 while in a packaged state of the intermittent-catheter assembly 800 or 900 for maintaining sterility of the intermittent catheter 10. The catheter tube 14 of the intermittent catheter 10 is exposed with the collapsible sheath 803 or 903 set forth below cinched up to the funnel 12 of the intermittent catheter 10 while in ready-to-used state of the intermittent-catheter assembly 800 or 900.

The catheter housing 802 or 902 includes at least a collapsible sheath 803 or 903 and a pull tab 831 or 931, which pull tab 831 or 931 may instead be considered packaging of the intermittent-catheter assembly 800 or 900 in the packaged state of the intermittent-catheter assembly 800 or 900. As to the intermittent-catheter assembly 800, the catheter housing 802 also includes an end piece 846 around the catheter tube 14 of the intermittent catheter 10 and a removable cap 812 sealing a proximal opening of the funnel 12 of the intermittent catheter 10 in the packaged state of the intermittent-catheter assembly 800. An end piece such as the end piece 846 is optional in the intermittent-catheter assembly 900. Also, instead of the removable cap 812 or the like, the intermittent-catheter assembly 900 includes another pull tab 930 for sealing the proximal opening of the funnel 12 and maintaining sterility of the intermittent catheter 10 in the packaged state of the intermittent-catheter assembly 900.

The collapsible sheath 803 or 903 includes a proximal portion coupled to the funnel 12 of the intermittent catheter 10. A distal portion of the collapsible sheath 803 is coupled to the end piece 846, which end piece, in turn, includes a distal opening sealed by the pull tab 831 in the packaged state of the intermittent-catheter assembly 800. In intermittent-catheter assemblies without an end piece such as the intermittent-catheter assembly 900 shown in FIG. 33, a distal opening of the collapsible sheath 903 is sealed by the pull tab 931 in the packaged state of the intermittent-catheter assembly 900. An entirety of the catheter tube 14 of the intermittent catheter 10 is disposed in the collapsible sheath 803 or 903 in the packaged state of the intermittent-catheter assembly 800 or 900, thereby maintaining sterility of the intermittent catheter 10.

As shown in FIG. 32B, the intermittent-catheter assembly 800 can include the drainage bag 22. The drainage bag 22 is fluidly coupled to the funnel 12 for voiding urine into the drainage bag 22 upon catheterization with the intermittent catheter 10. Notably, the cap 812 has a greater length in the intermittent-catheter assembly 800 of FIGS. 32A and 32B than in the intermittent-catheter assembly 800 of FIGS. 31A and 31B to accommodate the drainage bag 22.

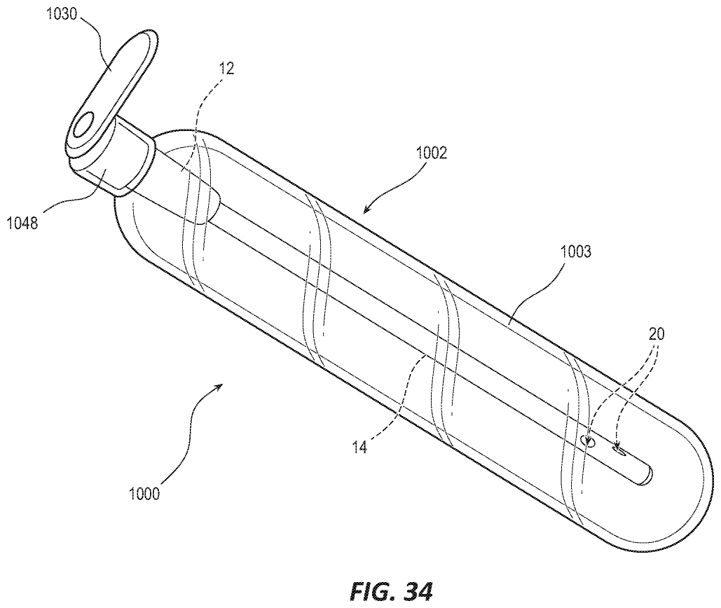

FIG. 34 illustrates a tenth intermittent-catheter assembly 1000 in a packaged state of the intermittent-catheter assembly 1000 in accordance with some embodiments.

As shown, the intermittent-catheter assembly 1000 includes the intermittent catheter 10 and a catheter housing 1002. The intermittent catheter 10 is disposed in the catheter housing 1002 while in the packaged state of the intermittent-catheter assembly 1000 for maintaining sterility of the intermittent catheter 10.

The catheter housing 1002 includes a bottle 1003. An entirety of the catheter tube 14 of the intermittent catheter 10 is disposed in the bottle 1003 with at least a portion of the funnel 12 of the intermittent catheter 10 fitted into a neck 1048 of the bottle 1003 in the packaged state of the intermittent-catheter assembly 1000. A proximal opening of the funnel 12 and a proximal opening of the bottle 1003 or the neck 1048 can be concentric in the packaged state of the intermittent-catheter assembly 1000. An inner diameter of the neck 1048 can be larger than an outer diameter of the funnel 12, thereby configuring the bottle 1003 to be fluidly coupled to the funnel 12 for voiding urine into the bottle 1003 upon catheterization with the intermittent catheter 10. Optionally, the funnel 12 can include a longitudinal channel or notch along a side thereof allowing therethrough an exchange of air in the bottle for urine voided from a bladder, particularly if the inner diameter of the neck 1048 is commensurate with the outer diameter of the funnel 12 and otherwise restricts fluid exchange.

A pull tab 1030 seals the intermittent catheter 10 in the bottle 1003 in the packaged state of the intermittent-catheter assembly 1000. When the proximal opening of the funnel 12 and the proximal opening of the bottle 1003 or neck 1048 are concentric, the pull tab 1030 simultaneously seals the funnel 12 and the bottle 1003 in the packaged state of the intermittent-catheter assembly 1000.

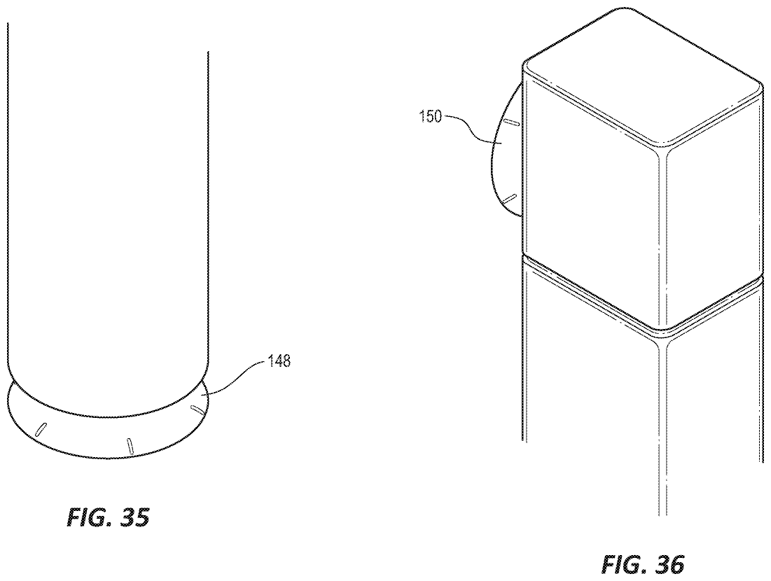

FIG. 35 and FIG. 36 respectively illustrate a bottom suction cup 148 and a side suction cup 150 for any intermittent-catheter assembly of those set forth herein. Such suction cups can be incorporated into a catheter housing (e.g., a sleeve or a cap) of an intermittent-catheter assembly for sticking the intermittent-catheter assembly or the catheter housing thereof to a surface (e.g., bathroom stall divider or door, top of a toilet, etc.) when desired or needed during catheterization. Such suction cups are particular useful for maintaining sterility in tight spaces such as bathroom stalls where available surface are limited or of questionable cleanliness.

### Methods

Methods of intermittent-catheter assemblies such as those set forth above include methods of using them. For example, a method of using any intermittent-catheter assembly of those set forth above includes one or more steps selected from a catheter assembly-obtaining step, an intermittent catheter-exposing step, an intermittent catheter-removing step, a catheter tube-inserting step, and a urine-voiding step.

For expository expediency, the foregoing steps are set forth in detail below with respect to the intermittent-catheter assemblies 100 and 800. However, the steps detailed below are set forth with the understanding that the steps can be practiced with other intermittent-catheter assemblies set forth above, albeit with certain modifications in view of the different configurations of the intermittent-catheter assemblies. As to such modifications, it should be understood that the intermittent-catheter assemblies or components thereof set forth above “configured to” or “configured for” effectuating some action often employ a user to effectuate that action.

The catheter assembly-obtaining step includes obtaining the intermittent-catheter assembly 100 or 800 in the packaged state of the intermittent-catheter assembly 100 or 800. The intermittent-catheter assembly 100 or 800 has the intermittent catheter 10 disposed in the catheter housing 102 or 802 in the packaged state of the intermittent-catheter assembly 100 or 800.

The intermittent catheter-exposing step depends upon the configuration of the intermittent-catheter assembly 100 or 800 used in practicing the method.

With respect to the intermittent-catheter assembly 100, the intermittent catheter-exposing step includes exposing the intermittent catheter 10 for removal from the catheter housing 102, which includes an outer sleeve-grasping step, a force-applying step, and an outer sleeve-sliding step. The outer sleeve-grasping step includes grasping the outer sleeve 106 of the catheter housing 102. The force-applying step includes applying a force to the exposed end of the inner sleeve 104 of the catheter housing 102. The outer sleeve-sliding step includes sliding the outer sleeve 106 toward the exposed end of the inner sleeve 104 in opposition to the force applied to the exposed end of the inner sleeve 104.

With respect to the intermittent-catheter assembly 800, the intermittent catheter-exposing step includes exposing the intermittent catheter 10 for insertion into a urethra, which includes a pull tab-removing step, an end piece-grasping step, and an end piece-sliding step. The pull tab-removing step includes removing the pull tab 831 sealing the distal opening of the end piece 846 of the catheter housing 802. The end piece-grasping step includes grasping the end piece 846. The end piece-sliding step includes proximally sliding the end piece 846 over the catheter tube 14 of the intermittent catheter 10 toward the funnel 12 of the intermittent catheter 10.

The intermittent catheter-removing step includes removing the intermittent catheter 10 from the catheter housing 102 or 802 after the intermittent catheter-exposing step. However, the intermittent catheter 10 of FIG. 32A need not be removed from the catheter housing 802.

The catheter tube-inserting step includes inserting the catheter tube 14 of the intermittent catheter 10 into a urethra.

The urine-voiding step includes voiding urine from a bladder with the intermittent catheter 10.

The method can further include a catheter tube-removing step and a catheter assembly-disposing step. The catheter tube-removing step includes removing the catheter tube 14 from the urethra after the urine-voiding step. The catheter assembly-disposing step includes disposing of the intermittent catheter 10 and the catheter housing 102 or 802. Optionally, the intermittent catheter 10 and the catheter housing 802 are disposed of in a reassembled state or partially reassembled state of the intermittent-catheter assembly 100 or 800 during the catheter assembly-disposing step.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An intermittent-catheter assembly, comprising:
an intermittent catheter including:
a funnel; and
a catheter tube fluidly coupled to the funnel; and
a catheter housing including the intermittent catheter disposed in the catheter housing in a packaged state of the intermittent-catheter assembly, the catheter housing including:
a sleeve including a longitudinal cavity containing a majority of the intermittent catheter in the packaged state of the intermittent-catheter assembly; and
a cap coupled to the sleeve by a living hinge, the cap sealing an opening of the sleeve opposite a closed end of the sleeve, thereby maintaining sterility of the intermittent catheter in the packaged state of the intermittent-catheter assembly.

2. The intermittent-catheter assembly of claim 1, wherein the opening includes a longitudinal extension into the sleeve configured to expose a longitudinal portion of the funnel when the cap is moved from a closed position in the packaged state of the intermittent-catheter assembly to an open position about the living hinge.

3. The intermittent-catheter assembly of claim 1, wherein the cap includes a pull tab coupled to the cap on a same side of the catheter housing as the living hinge, a portion of the pull tab configured to peel away from the cap toward an opposite side of the catheter housing from the living hinge when the pull tab is initially pulled to extend a lever arm with respect to the living hinge sufficient for subsequently pulling the cap away from the opening of the sleeve by the pull tab.

4. The intermittent-catheter assembly of claim 1, wherein the cap includes a push tab extending from an opposite side of the catheter housing from the living hinge, the push tab configured to extend a lever arm with respect to the living hinge sufficient for pushing the cap away from the opening of the sleeve by the push tab.

5. The intermittent-catheter assembly of claim 1, wherein the cap includes a push button configured to deform the cap and disengage complementary snap-fit features between the cap and the sleeve for subsequently pushing the cap away from the opening of the sleeve by the push button.

6. The intermittent-catheter assembly of claim 1, further comprising a drainage bag fluidly coupled to the funnel for voiding urine into the drainage bag upon catheterization with the intermittent catheter.

7. An intermittent-catheter assembly, comprising:
an intermittent catheter including:
a funnel; and
a catheter tube fluidly coupled to the funnel; and
a catheter housing including the intermittent catheter disposed in the catheter housing in a packaged state of the intermittent-catheter assembly, the catheter housing including:
a sleeve including a longitudinal cavity containing a majority of the intermittent catheter in the packaged state of the intermittent-catheter assembly; and
a removable cap including another longitudinal cavity containing a remainder of the intermittent catheter in the packaged state of the intermittent-catheter assembly, the cap sealing an opening of the sleeve opposite a closed end of the sleeve, thereby maintaining sterility of the intermittent catheter in the packaged state of the intermittent-catheter assembly.

8. The intermittent-catheter assembly of claim 7, wherein the sleeve includes longitudinal ribs extending into the longitudinal cavity, the ribs stabilizing the intermittent catheter in the sleeve in the packaged state of the intermittent-catheter assembly.

9. The intermittent-catheter assembly of claim 7, wherein the sleeve is approximately coextensive with the catheter tube and the cap is approximately coextensive with the funnel.

10. The intermittent-catheter assembly of claim 7, wherein the cap and the sleeve include complementary snap-fit features securing the cap on the sleeve in the packaged state of the intermittent-catheter assembly.

11. The intermittent-catheter assembly of claim 7, further comprising shrink-wrap packaging over an entirety of the cap and at least a portion of the sleeve, the packaging including a pull tab extending from the packaging configured to break open the packaging when the pull tab is pulled.

12. The intermittent-catheter assembly of claim 7, wherein the cap includes internal threads about an open-ended portion of the cap and the sleeve includes complementary external threads about an open-ended portion of the sleeve including the opening, the cap including recesses or ribs around the cap configured to facilitate gripping and screwing the cap off the sleeve.

13. The intermittent-catheter assembly of claim 7, further comprising a drainage bag fluidly coupled to the funnel for voiding urine into the drainage bag upon catheterization with the intermittent catheter.

14. An intermittent-catheter assembly, comprising:
an intermittent catheter including:
a funnel; and
a catheter tube fluidly coupled to the funnel; and
a catheter housing including the intermittent catheter disposed in the catheter housing in a packaged state of the intermittent-catheter assembly, the catheter housing including:
a sleeve including a longitudinal cavity containing a majority of the intermittent catheter in the packaged state of the intermittent-catheter assembly; and
a reinforcing insert suspended in a longitudinal sleeve gap, the reinforcing insert including a longitudinal insert gap such that the sleeve gap and the insert gap combine to provide major-side openings for grasping the funnel and removing the intermittent catheter from the catheter housing; and
an adhesive tab covering the major-side openings of the catheter housing.

15. The intermittent-catheter assembly of claim 14, wherein the reinforcing insert includes major-side protrusions from which the reinforcing insert is suspended in the sleeve gap.

16. The intermittent-catheter assembly of claim 14, wherein the reinforcing insert includes minor-side protrusions from which the reinforcing insert is suspended in the sleeve gap.

17. The intermittent-catheter assembly of claim 14, further comprising a drainage bag fluidly coupled to the funnel for voiding urine into the drainage bag upon catheterization with the intermittent catheter.

18. An intermittent-catheter assembly, comprising:
an intermittent catheter including:
a funnel; and
a catheter tube fluidly coupled to the funnel; and
a catheter housing including the intermittent catheter disposed in the catheter housing in a packaged state of the intermittent-catheter assembly for maintaining sterility of the intermittent catheter, the catheter housing including:
an end piece;
a collapsible sheath including a distal portion coupled to the end piece and a proximal portion coupled to the funnel, an entirety of the catheter tube disposed in the collapsible sheath in the packaged state of the intermittent-catheter assembly;
a pull tab sealing a distal opening of the end piece in the packaged state of the intermittent-catheter assembly; and
a removable cap sealing a proximal opening of the funnel in the packaged state of the intermittent-catheter assembly.

19. The intermittent-catheter assembly of claim 18, further comprising a drainage bag fluidly coupled to the funnel for voiding urine into the drainage bag upon catheterization with the intermittent catheter.

20. An intermittent-catheter assembly, comprising:
an intermittent catheter including:
a funnel; and
a catheter tube fluidly coupled to the funnel; and
a catheter housing including the intermittent catheter disposed in the catheter housing in a packaged state of the intermittent-catheter assembly for maintaining sterility of the intermittent catheter, the catheter housing including:
a bottle including a neck, an entirety of the catheter tube disposed in the bottle with the funnel fitted into the neck in the packaged state of the intermittent-catheter assembly; and
a pull tab sealing the intermittent catheter in the bottle in the packaged state of the intermittent-catheter assembly.

21. The intermittent-catheter assembly of claim 20, wherein a proximal opening of the funnel and a proximal opening of the bottle are concentric such that the pull tab simultaneously seals the funnel and the bottle in the packaged state of the intermittent-catheter assembly.

22. The intermittent-catheter assembly of claim 20, wherein the bottle is configured to be fluidly coupled to the funnel for voiding urine into the bottle upon catheterization with the intermittent catheter.
